(19) United States
(12) Patent Application Publication
(10) Pub. No.: US 2019/0076116 A1
(43) Pub. Date: Mar. 14, 2019

(54) MAGNETICALLY DRIVEN ULTRASONIC SCANNING DEVICE

(71) Applicants: National Yang-Ming University, Taipei City (TW); Portwell, Inc., New Taipei City (TW)

(72) Inventors: Hui-Hua CHIANG, Taipei City (TW); Yin-Chih LIN, Taipei City (TW)

(21) Appl. No.: 16/130,618

(22) Filed: Sep. 13, 2018

(30) Foreign Application Priority Data
Sep. 14, 2017 (TW) 106131626

(51) Int. Cl. A61B 8/00; G01S 7/52; B06B 1/06

(52) U.S. Cl. CPC A61B 8/4218; B06B 1/0622; G01S 7/52085

(57) ABSTRACT

The present invention provides an ultrasonic probe scanning device comprising: a disposable part and a fixed part. In the disposable part, an ultrasonic probe with a magnetic end attaches to a slide rail encapsulated in a water-containing closed area. Otherwise, in the fixed part, a magnetic holder attached to a track, and a motor for driving the magnetic holder to move back and forth on the track. When the disposable part and the fixed part be combined, it can drive the motion of ultrasonic scanning device by magnetic attraction force.

### CROSS-REFERENCE TO RELATED APPLICATIONS

This Non-provisional application claims priority under 35 U.S.C. § 119(a) on Patent Application No(s). [106131626] filed in Taiwan, Republic of China [Sep. 14, 2017], the entire contents of which are hereby incorporated by reference.

### FIELD OF THE INVENTION

The present invention s an ultrasonic scanning device being used for the skin surface image and the subcutaneous tissue. Especially, a device is with a moveable ultrasonic probe head driven by magnetic attraction force.

### BACKGROUND OF THE INVENTION

Because of no-radioactivity and safety, the application of ultrasound technique in medicine is more and more widely in recent year. A part of the application is focused on the development of medical, such as obstetric ultrasound, breast ultrasound, muscle ultrasound . . . etc. In addition to diagnostic use, it can also be used for therapentic treatment, such as ultrasound scalpel, ultrasound heat treatment, ultrasound lifting and lipolysis.

In the traditional ultrasound device for skin and subcutaneous tissue use, the ultrasonic probe is in one end of the device, the linear shaft rod is in the middle to drive the device, and the linear shaft is designed as the mechanical structure, which is covered by a rubber tube. However, the rubber tube limits the movement distance during actual operation. Otherwise, the rubber tube is often a leaky spot which causes the damage of the whole product. It is difficult to maintain the quality of ultrasound device product. Moreover, the size of the linear shaft bar cannot continue to be reduced.

For solving above problem, the present invention is to replace the conventional mechanical structure with magnetic attraction force and a detachable model.

### SUMMARY OF THE INVENTION

Accordingly, the present invention provides a design for driving a imaging probe and a therapy probe, having the following features:

1. The design of Ultrasonic probe design is with high collimation;

2. The ultrasonic probe can move stably through the magnetic attraction force;

3. A disposable part with the probe can be separated from a fixed part with the motor, and its water seal design can also reduce the risk of water seepage.

The ultrasonic probe scanning device comprising a disposable part and a fixed part. Wherein the disposable part having: a power input port; a slide rail; and an ultrasonic probe having a magnetic end with at least a pair of magnet N and S poles, wherein the ultrasonic probe is connected to the slide rail and electrically connected to the power input port, wherein the slide rail and the ultrasonic probe are encapsulated in a water-containing closed area.

The fixed part is having: a power output slot for electrically engaging the power input port when the disposable part is combined with the fixed part; a track; a magnetic holder coupled to the track for magnetically driving the ultrasonic probe when the disposable part is combined with the fixed part; and a motor which drives the magnetic holder to move on the track.

### BRIEF DESCRIPTION OF THE DRAWINGS

FIG. 1A and FIG. 1B is the exterior picture of ultrasonic probe scanning device of the present invention.

FIG. 2 is the internal structure view of ultrasonic probe scanning device of the present invention.

FIG. 3A and FIG. 3B show the front and back view of an ultrasonic probe of the present invention.

FIG. 4 shows the test result of magnetic attractive force.

### DETAILED DESCRIPTION OF THE INVENTION

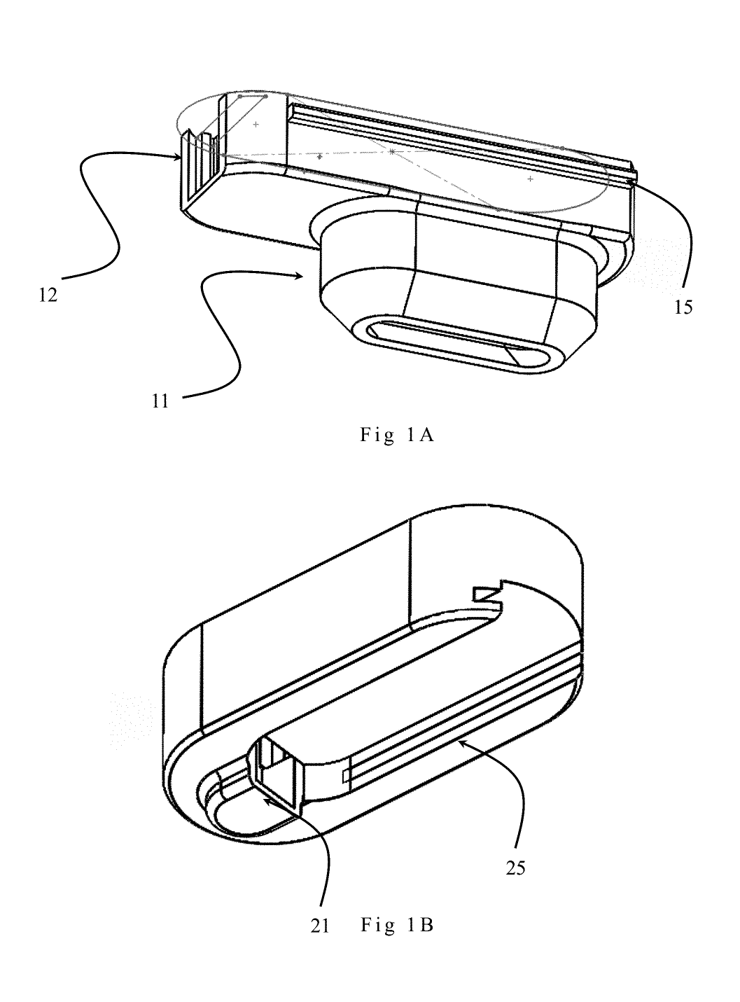

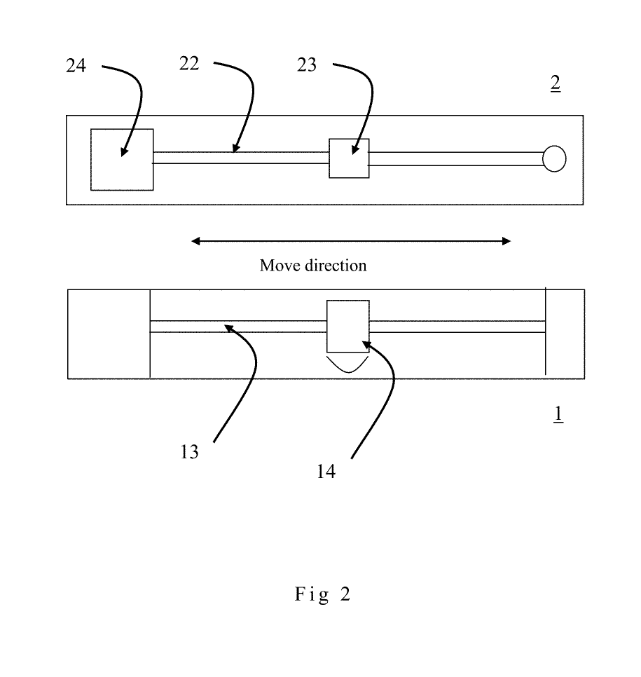

The design of this ultrasonic probe scanning device shows as FIG. 1 and FIG. 2, which comprises: a disposable part 1 and a fixed part 2.

The FIG. 1A is the exterior picture of disposable part 1, and FIG. 2 is the internal structure view of disposable portion 1 and the fixed portion 2, wherein the disposable part 1 comprises: a power input port 12; a slide rail 13; and an ultrasonic probe 14 having a magnetic end having at least a pair of magnet N and S poles, wherein the ultrasonic probe 14 is connected to the slide rail 13 and electrically connected to the power input port 12, wherein the slide rail 13 and the ultrasonic probe 14 are encapsulated in a water-containing closed area 11;

The FIG. 1B is the exterior picture of the fixed part 2, wherein the fixed part 2 having: a power output slot 21 for electrically engaging the power input port 21 when the disposable part 1 is combined with the fixed part 2; a track 22; a magnetic holder 23 coupled to the track 22 for magnetically driving the ultrasonic probe 14 when the disposable part 1 is combined with the fixed part 2; and a motor 24 which drives the magnetic holder 23 to move on the track 22.

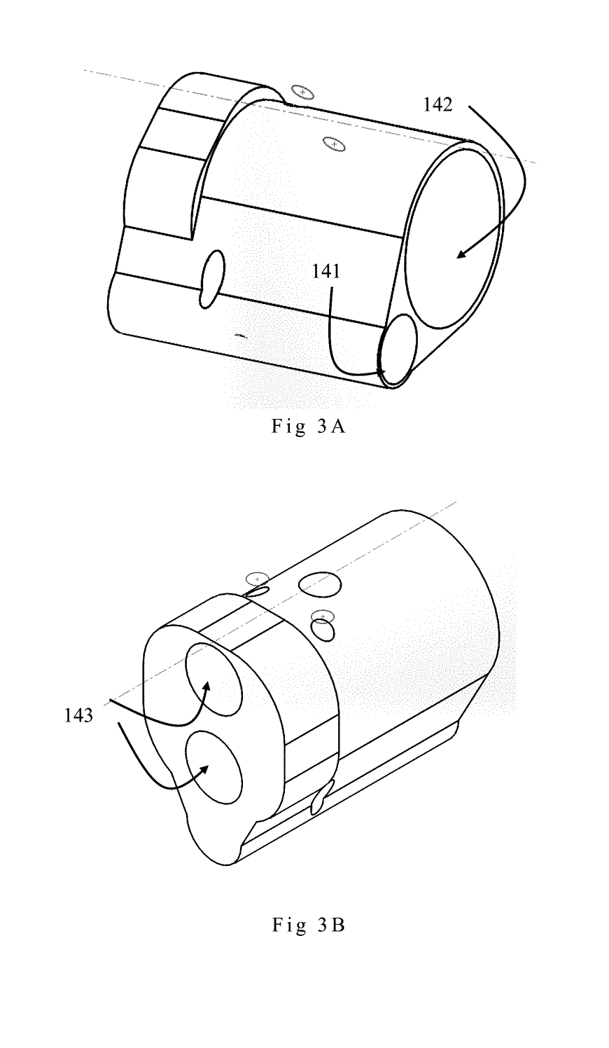

In one of the best embodiment, FIG. 3A shows the front view of the ultrasonic probe 14, wherein the ultrasonic probe 14 comprises a therapy probe 141; and an imaging probe 142, which is parallel to the irradiation direction of the therapy probe 141. In one of the best embodiment, the therapy probe 141 may be designed in the center of the imaging probe 142 or the therapy probe 141 may be designed on the outside of the imaging probe 142, the imaging probe 142 should have the same focal plane as the therapy probe 141; preferably, the operating frequency of the imaging probe 142 is 5 to 20 MHz, and the operating frequency of the therapy probe 141 is 2 to 10 MHz.

The ultrasonic probe 14 further comprises a miniature brake, which is used for controlling the ultrasonic probe 14 moving perpendicularly to the slide rail 13, when the target position is different to the preset focal plane, the user can adjust the focal plane distance of miniature brake so as to obtain a clearer image.

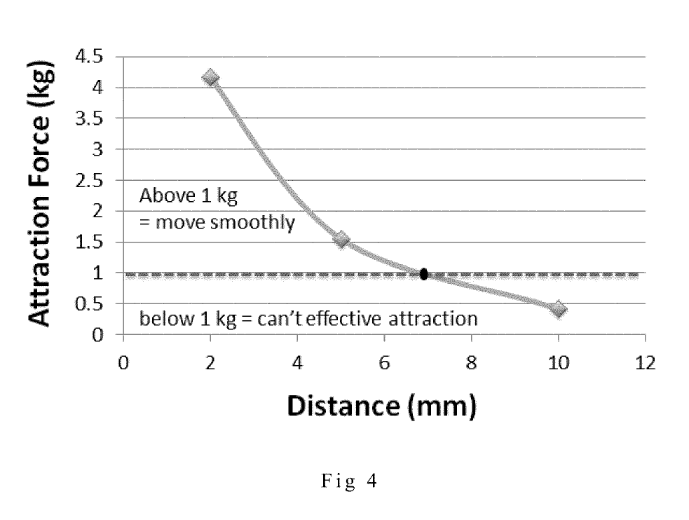

FIG. 3B shows the rear view of the ultrasonic probe 14, the fixing part 2 drives the disposable part 1 through the magnetic end 143 of the ultrasonic probe 14 in the present invention. For the ultrasonic probe 14 can cooperate with the motor 24 action. The present invention further calculates the relationship between the distance and magnetic attractive force, please refer to the following table 1-3 and FIG. 4, to evaluate the operational feasibility according to calculate the strength of the magnetic force.

TABLE 1
The test result of magnetic attractive force (distance is 5 mm)

| | | | magnetic attractive |
|---|---|---|---|
| magnetic base | magnetic end | distance | force |
| (thickness mm) | (thickness mm) | (mm) | (kg) |
| 5 | 10 | 5 | 1.548 |
| 5 | 5 | 5 | 0.774 |
| 5 | 2 | 5 | 0.306 |
| 5 | 1 | 5 | 0.1548 |

TABLE 2
The test result of magnetic attractive force (distance is 2 mm)

| | | | magnetic attractive |
|---|---|---|---|
| magnetic base | magnetic end | distance | force |
| (thickness mm) | (thickness mm) | (mm) | (kg) |
| 5 | 10 | 2 | 4.158 |
| 5 | 5 | 2 | 2.079 |
| 5 | 2 | 2 | 0.828 |
| 5 | 1 | 2 | 0.4158 |

TABLE 3
The test result of magnetic attractive force (distance is 10 mm)

| | | | magnetic attractive |
|---|---|---|---|
| magnetic base | magnetic end | distance | force |
| (thickness mm) | (thickness mm) | (mm) | (kg) |
| 5 | 10 | 10 | 0.414 |
| 5 | 5 | 10 | 0.207 |
| 5 | 2 | 10 | 0.0828 |
| 5 | 1 | 10 | 0.0414 |

When the thickness of the magnet is greater than 2 mm, the diameter is greater than 10 mm, and the spacing is less than 6 mm, the magnetic attractive force will be greater than 1 kg. Otherwise, the magnetic attractive force will be greater than the load of the probe holder, which is in the range of magnetic attractive force. Thus, the ultrasonic probe is drove by the motor and the magnetic base described above, and the magnetic attractive force causes the probe to automatically collimate.

In one of the best embodiment, the magnetic attraction between the magnetic end 15 and the magnetic base 23 is greater than 0.2 kg and the distance is less than 1 cm.

In the case of using the ultrasonic probe scanning device, because of the disposable part 1 is detachable, it can combine a new disposable part 1 if the disposable part needs to be cleaned or is damaged, and the power input port 12 is electrically connected to the power output slot 21, therefore, the power output slot 21 supplies the power source of the ultrasonic probe 14 and controls the operation of the ultrasonic probe 14. Preferably, the disposable part 1 and the fixed part 2 have an engagement groove respectively 15, 25.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### SYMBOLS

disposable part 1
closed area 11
power input port 12
slide rail 13
ultrasonic probe 14
engagement groove of disposable part 15
therapy probe 141
imaging probe 142
magnetic end 143
fixed part 2
power output slot 21
track 22
magnetic base 23
motor 24
engagement groove of fixed part 25

What is claimed is:

1. An ultrasonic probe scanning device comprising:
a disposable part having:
a power input port;
a slide rail: and
an ultrasonic probe having a magnetic end with at least a pair of magnet N and S poles, wherein the ultrasonic probe is connected to the slide rail and electrically connected to the power input port, wherein the slide rail and the ultrasonic probe are encapsulated in a water-containing closed area;
a fixed part having:
a power output slot for electrically engaging the power input port when the disposable part is combined with the fixed part;
a track;
a magnetic holder coupled to the track for magnetically driving the ultrasonic probe when the disposable part is combined with the fixed part; and
a motor which drives the magnetic holder to move on the track.

2. The ultrasonic probe scanning device of claim 1, wherein the ultrasonic probe further comprises a miniature brake, which is used for controlling the ultrasonic probe moving perpendicularly to the slide rail.

3. The ultrasonic probe scanning device of claim 1, wherein the ultrasonic probe comprises:
a therapy probe; and
an imaging probe, which is parallel to the irradiation direction of the therapy probe.

4. The ultrasonic probe scanning device of claim 3, wherein the imaging probe has the same focal plane as the therapy probe.

5. The ultrasonic probe scanning device of claim 3, wherein the operating frequency of the imaging probe is 5 to 20 MHz.

6. The ultrasonic probe scanning device of claim 3, wherein the operating frequency of the therapy probe is 2 to 10 MHz.

7. The ultrasonic probe scanning device of claim 1, wherein the suction force between the magnetic end and the magnetic base is greater than 0.2 kg and the pitch thereof is less than 1 cm.

8. The ultrasonic probe scanning device of claim 1, wherein the disposable part and the fixed part each have a mutual engagement groove.
